(19) United States
(12) Patent Application Publication
(10) Pub. No.: US 2005/0171584 A1
(43) Pub. Date: Aug. 4, 2005

(54) Heating devices and apparatuses employing far infrared radiation and negative ions

(76) Inventor: Fred M. Slingo, Barrington, IL (US)

Correspondence Address: WILLIAM J. LENZ, 311 S. WACKER DRIVE, 53RD FLOOR, CHICAGO, IL, 60606-6622, US

(21) Appl. No.: 10/770,156

(22) Filed: Feb. 2, 2004

(51) Int. Cl. A61F 7/00; A61F 7/12

(52) U.S. Cl. 607/96

(57) ABSTRACT

Heating devices and apparatuses and methods of manufacturing and using same are provided. The devices and apparatuses include a powered heating element and a material so configured and arranged to allow the material to be effectively heated so as to emit both a infrared radiation and negative ions.

### BACKGROUND OF THE INVENTION

The present invention relates to devices and apparatuses for heating and methods of manufacturing and using same. More specifically, the present invention relates to heating devices and apparatuses and methods of using and manufacturing same that employ a material or combination of materials capable of emitting both far infrared radiation (FIR) and negative ions.

FIR commonly refers to electromagnetic radiation that has a wavelength between the visible light region and the microwave region of the electromagnetic spectrum. FIR-emitting bodies have been used in general, with respect to a variety of applications, such as increasing fuel efficiency, heating, ripening of fruit, deodorizing, alleviating pain, and inducing perspiration. With respect to fuel efficiency applications, devices have been developed that can introduce FIR to fuel prior to introduction of the fuel into an engine. FIR has also been used in saunas as a substitute for traditional steam heat.

In recent years, FIR, in general, has been used in a therapeutic manner. In this regard, water molecules resonate when exposed to FIR. This may improve blood circulation and thus promote health. As a consequence, a variety of products that have FIR-emitting properties are commercially available.

Certain types of ceramics containing silica oxide and aluminum oxide are known to radiate FIR at room temperature, and to radiate elevated levels of FIR when heated. Some of these ceramics are commonly referred to as bio-ceramics because of reported biological and physiological effects attributed to such materials. However, typical bio-ceramic materials do not emit negative ions at room temperature and are generally limited in their ability to do so unless subjected to temperatures exceeding about 1000° F.

Materials are also known that are capable of emitting negative ions. These types of materials, in general, have industrial as well as consumer applications. For example, materials that can emit negative ions have been reported to possess antibacterial and deodorizing properties, improve the flavor and taste of drinking water, and be effective in removal of airborne pollutants. However, the application of negative ion technology can be problematic. For example, the application of this technology typically can require extensive modifications to existing processes, thus increasing costs associated with those processes.

Other materials are known, in general, that can emit both FIR and negative ions. For example, U.S. Pat. No. 6,402,991 discloses a function-enhanced shaped ceramic article obtained by mixing a powder of a functional material of at least one species selected from the group consisting of a mineral, a metal and metallic compound and a powder of a FIR material composed of a ceramic composition that contains SiO₂ and Al₂O₃ in specified amounts. Preferably, the amount of the far infrared radiating material is at least 30% weight and not more than 90% weight. As disclosed, this amount of far infrared radiating material is necessary for the article to be easily shaped into a plate-like or a ball-like shaped article. The function-enhanced shaped ceramic article can be directly added to water for anti-bacterial purposes as disclosed in Example 1 of U.S. Pat. No. 6,402,991.

U.S. Pat. No. 5,965,007 discloses a method of preparing water for human consumption and/or use. As disclosed, the method includes submerging ceramics that have an extremely high emissivity of far infrared in water in a specified amount; placing an electrode in the water; and allowing the water to stand for a predetermined period of time, preferably at least 12 hours. The ceramics have such a composition that SiO₂, Al₂O₃, Fe₂O₃, MnO₂, ZnO and CoO are provided in specified concentrations.

A need, therefore, exists to provide improved devices, apparatuses, and methods that utilize the emission of FIR and negative ions, such as for therapy or treatment purposes.

### SUMMARY OF THE INVENTION

The present invention, in general, relates to heating devices and apparatuses and methods of making and using same. In particular, the present invention relates to heating devices and apparatuses that include a material capable of emitting both far infrared radiation and negative ions, and methods of manufacturing and using same.

The present invention can provide improved heating conditions, particularly as applied in a therapeutic manner to an individual in need of same. In this regard, the present invention can enhance the effectiveness of heat therapy by also subjecting the individual to an effective amount of both negative ions and FIR emitted by the material or combination of materials.

To this end, in an embodiment, the present invention provides a device capable of emitting both far infrared radiation and negative ions. The device includes a powered heating element and a material adaptedly coupled to the powered heating element thereby allowing the material to be effectively heated so as to emit both far infrared radiation and negative ions in a therapeutic manner.

In an embodiment, the powered heating element is integral with a cover that incorporates the powered heating element and the material.

In an embodiment, the powered heating element is integral with the material capable of emitting both far infrared radiation and negative ions.

In an embodiment, the material capable of emitting both far infrared radiation and negative ions is integral with a cover that surrounds the powered heating element.

In an embodiment, the material includes a single material having one or more constituents and combination of two or more materials.

In an embodiment, the material includes a first part including a bio-ceramic in an amount of about 95% by weight or less and a second part including a ceramic oxide in an amount of about 5% by weight or more.

In an embodiment, the first part includes at least one compound that includes silicon oxide, aluminum oxide, iron oxide, magnesium oxide, the like, derivatives thereof and combinations thereof.

In an embodiment, the ceramic oxide includes iron oxide, silicon oxide, titanium oxide, aluminum oxide, magnesium oxide, the like, derivatives thereof and combinations thereof.

In another embodiment, an apparatus capable of emitting both far infrared radiation and negative ions is provided. The apparatus includes a cover; a powered heating element wherein the heating element is adaptedly coupled to the cover; and a flexible material capable of emitting both far infrared radiation and negative ions wherein the flexible material is adaptedly coupled to the powered heating element and the cover allowing, the material to be heated, and wherein the flexible material includes a first part including a bio-ceramic in an amount of about 95% by weight or less and a second part including a ceramic oxide in an amount of about 5% by weight or more.

In an embodiment, the powered heating element is integral with the cover.

In an embodiment, the apparatus is configured as a heating apparatus for therapeutic applications selected from the group consisting of a heating pad, a heating wrap, a heating collar, a heating cuff, a heating patch, a heating blanket, the like and combinations thereof.

In yet another embodiment, the present invention provides a method of providing heat therapy to an individual in need of same. The method includes the steps of providing a heating apparatus, the heating apparatus including a cover, a powered heating element, and a material capable of emitting both far infrared radiation and negative ions; applying the heating apparatus to at least a portion of a body part of the individual; supplying power to the heating element; and heating the material sufficiently to release an amount of far infrared radiation and negative ions to provide effective heat therapy to the individual.

In an embodiment, the heating element includes a wire, a coil, a pad, a resistor, an exothermic chemical composition, the like and combinations thereof.

In an embodiment, power is supplied to the heating element via at least one of an external power source and an internal power source.

In an embodiment, the body part includes an arm region, an elbow region, a knee region, a back region, a head region, a neck region, a foot region, a hand region, a forearm region, a shoulder region, a wrist region, an ankle region, the like and combinations thereof.

In an embodiment, the far infrared radiation and negative ions are emitted in a sufficient amount to penetrate the body part to a depth ranging from about 25 mm to about 35 mm.

In still yet another embodiment, the present invention provides a method of manufacturing a heating apparatus. The method includes providing a powered heating element and a material capable of emitting both far infrared radiation and negative ions; and adaptedly coupling the powered heating element and the material to form the heating apparatus.

An advantage of the present invention is to provide improved devices and apparatuses for heating and improved methods of manufacturing and using same.

Another advantage of the present invention is to provide improved heating devices and apparatuses that are capable of emitting both far infrared radiation and negative ions.

Still another advantage of the present invention is to provide improved heat therapy to a person in need of same.

Yet still another advantage of the present invention is to provide improved heating devices and apparatuses that can be made with relative ease and at reduced costs.

Additional features and advantages of the present invention are described in, and will be apparent from, the following Detailed Description of the Invention and the figures.

### BRIEF DESCRIPTION OF THE FIGURES

FIG. 1 is a sectional view of a device according to an embodiment of the present invention.

FIG. 2 is a sectional view of a device according to an embodiment of the present invention.

FIG. 3 is a sectional view of an apparatus as shown applied to a body part of an individual according to an embodiment of the present invention.

FIG. 4 is a sectional view of an apparatus as shown wrapped around a body part of an individual according to an embodiment of the present invention.

FIG. 5 is a sectional view of an apparatus according to an embodiment of the present invention.

FIG. 6 is a sectional view of an apparatus according to an embodiment of the present invention.

FIG. 7 is a sectional view of an apparatus according to an embodiment of the present invention.

FIG. 8 is a sectional view of an apparatus according to an embodiment of the present invention.

FIG. 9 is a sectional view of an apparatus according to an embodiment of the present invention.

### DETAILED DESCRIPTION OF THE INVENTION

The present invention generally relates to devices and apparatuses for heating and methods of making and using same. More specifically, the present invention relates to heating devices and apparatuses that include a material capable of emitting both FIR and negative ions, and methods of manufacturing and using same, preferably in therapeutic applications.

It is believed that the present invention can enhance the effectiveness of heat therapy by exposing an individual to an effective amount of both negative ions and FIR emitted by the material or combination of materials according to an embodiment of the present invention. Far infrared radiation is an energy source that can be characterized by its specific electromagnetic wave properties ranging from about 5.6 microns to about 1000 microns, preferably ranging from about 5.6 microns to about 25 microns. Negative ions, which are essentially negatively charged particles, are also an energy form.

In this regard, it is believed that the two part material energy source, or combination of energy sources, capable of emitting both FIR and negative ions can be adapted to have an enhanced therapeutic effect, thereby promoting health. Further, the present invention can be made with relative ease and can be adapted for use with relatively minor modifications to the existing heating apparatuses, thus keeping costs at a minimum.

As previously mentioned, the present invention provides heating devices and apparatuses that include a material or a combination of materials capable of emitting both FIR and negative ions. In an embodiment, the material includes a first part capable of emitting FIR and a second part that includes an oxide material. Applicants believe that the combination of these material components provides improved properties as compared to known applications of ceramic materials.

In an embodiment, the material at least includes a first part and a second part. The first part and second part of the material of the present invention can be made of a variety of suitable materials. In an embodiment, the FIR-emitting material of the first part is composed of a bio-ceramic material. The bio-ceramic material can include, for example, silicon oxide (SiO₂), aluminum oxide (Al₂O₃), iron oxide (Fe₂O₃), magnesium oxide (MgO) and other suitable constituents, derivatives thereof or combinations thereof. These materials are commercially available or can be manufactured in any known and suitable way. It should be appreciated that the bio-ceramic material can include any suitable amount of the constituents.

The second part or additional oxide material includes, in an embodiment, iron oxide, silicon oxide, titanium oxide (TiO₂), aluminum oxide, magnesium oxide and other suitable materials, derivatives thereof or combinations thereof. The additional oxide material is commercially available. The additional oxide materials can also be manufactured according to known procedures. It should be understood, however, that the ratio of bio-ceramic material to the additional oxide material can vary depending on the desired application. In an embodiment, the material of the present invention includes about 95% by weight or less of the bio-ceramic material and about 5% by weight or more of the additional oxide material. Preferably, the bio-ceramic part includes about 70% by weight or more of silicon oxide, about 20% by weight or more of aluminum oxide, about 3% by weight or more of iron oxide, and about 2% by weight or more of magnesium oxide.

It should be appreciated that the emitting material of the present invention can be processed into a variety of different and suitable sizes, including the production of small-gauge particulates or powders. In general, as the particle size of the material is decreased and/or the surface area is increased, the emitting properties of the present invention can be optimized. While the far infrared and negative ion emitting substance is a necessary component of the present invention, other materials optionally can be mixed with or added to the emitting substance. Other such optional substances may include, for example, binders, fillers and/or other suitable material components.

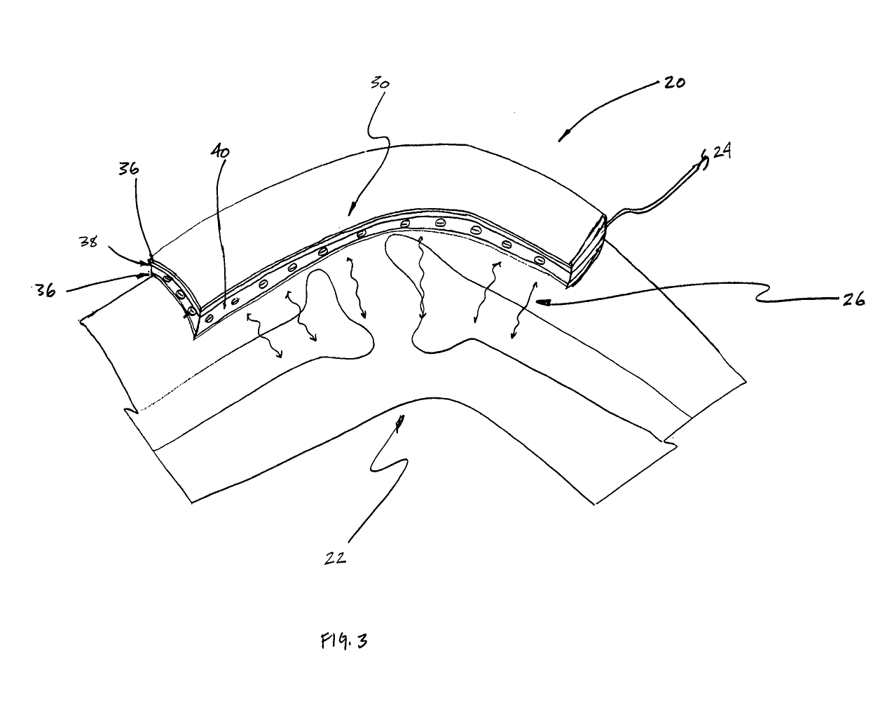

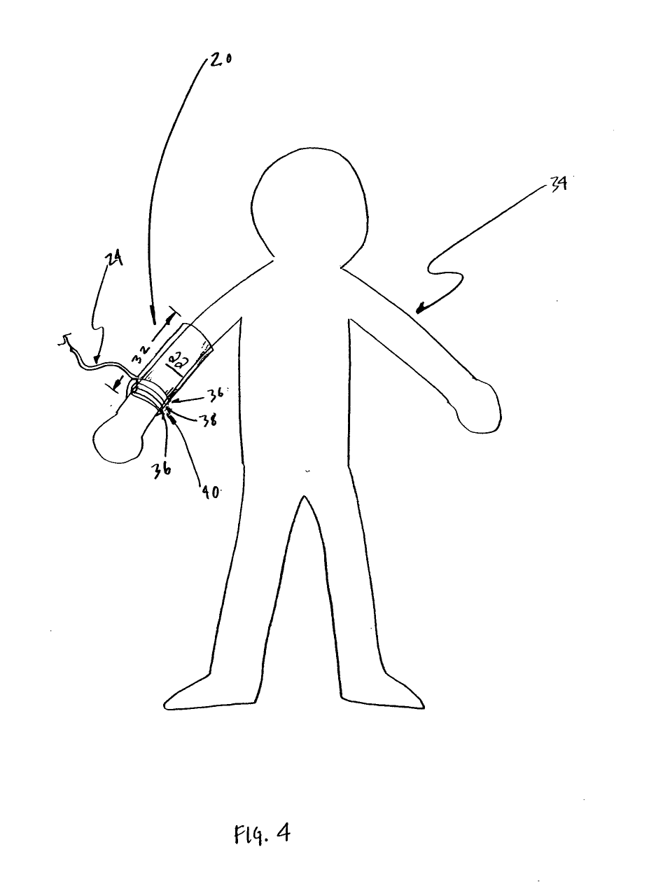

The emitting substances can be attached to, or incorporated with, a variety of other substances that will serve as substrates or supports to facilitate adaptation of the material for use. For example, the emitting substance can be processed into a powder/particulate form and adhered to or incorporated onto, such as by a bonding agent including adhesives, glues and/or the like, a flexible substrate as described below and shown in FIGS. 3 and 4. While it is preferred that such a flexible substrate be made of cloth or other like textile material, other flexible, wrappable, and/or moldable materials can include, for example, rubber, plastic, other suitable polymeric substances and/or the like. If a flexible substrate is employed, it may be desirable to use a substrate that has elastomeric properties as this may allow for the device to be more securely and easily placed in position, such as wrapped around at least a portion of a body part, such as an arm region.

If a polymer-based material or the like is used, the FIR and negative ion emitting material, in solid and/or powder form, can be adhered to the substrate as previously discussed. Further, in powder form, the FIR and negative ion emitting material can be mixed with components of the polymer-based material during processing thereof, thus incorporating the FIR and negative ion material onto the substrate material. The substrate incorporated with the FIR and negative ion emitting material can be used as a stand-alone component part of the heating device and apparatus of the present invention. Further, it can be used to house the heating element and/or act as a cover for the devices and apparatuses of the present invention depending on the application. It should be appreciated that the component parts of the heating devices and apparatuses of the present invention (i.e., cover, heating element, FIR/negative ion emitting material, etc..) can be configured and arranged in any suitable manner, examples of which are described below in greater detail without limitation to the scope of the present invention.

Far infrared radiation and negative ion emitting substances can also be adhered to and/or incorporated in any suitable manner substrate materials that are more rigid as compared to flexible substrates described above depending on the application. A number of suitable rigid materials of varying shapes and sizes can be used that are composed of, for example, metals, high viscosity polymeric materials, and/or the like.

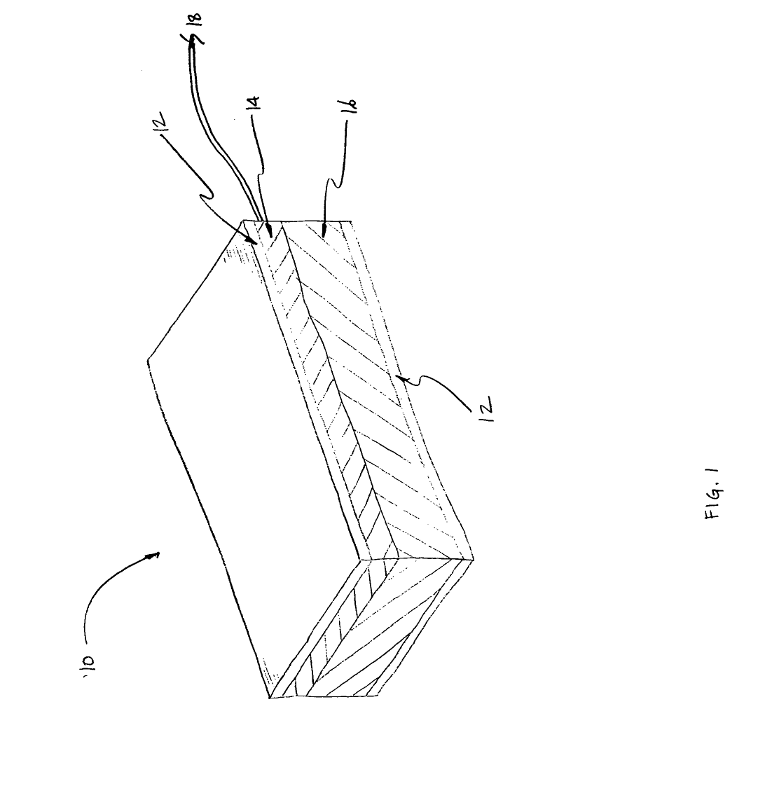

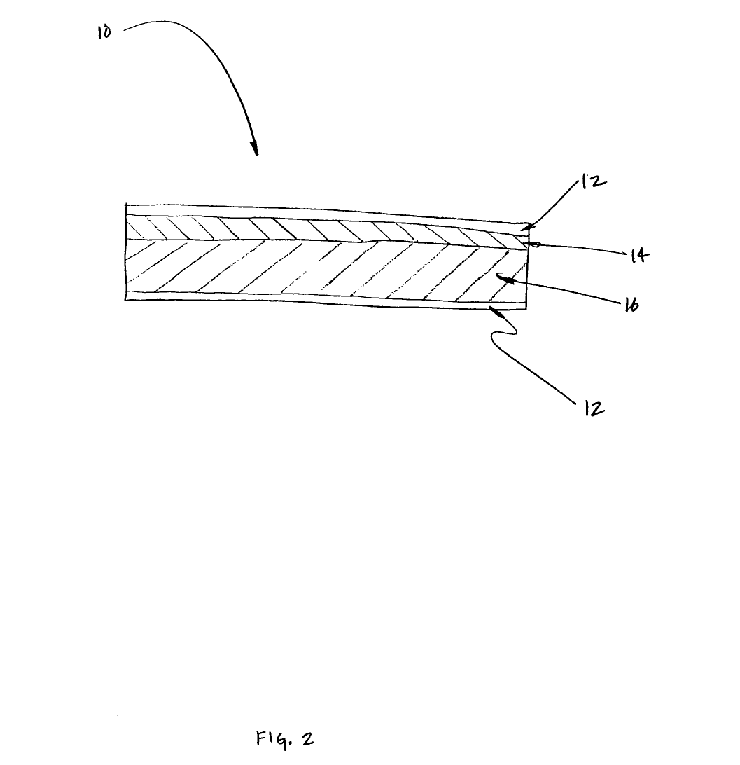

FIGS. 1 and 2 show sectional views of a heating device 10 according to an embodiment of the present invention. As shown, a cover 12 is provided. The cover 12 can be made of any suitable material, preferably a substantially flexible material. The heating device 10 also includes a powered heating element 14. The heating element 14 can include, for example, a wire, a coil, a pad, a resistor, an exothermic chemical composition, other suitable heating elements including commercially-available products, and combinations thereof (not shown). It should be appreciated that any suitable heating element 14 can be used. The device 10 further includes a far infrared radiation and negative ion emitting material 16. In FIG. 1, the powered heating element 14 includes a power cord 18 that connects to a power source (not shown). In FIG. 2, the powered heating element 14 includes a battery and/or other suitable power source (not shown) and thus is powered in a cordless manner. It should be appreciated that the cover 12, heating element 14 and the material 16 can be so configured and constructed in any suitable manner. For example, the cover 12 can be fitted to surround the material 16 and the heating element 14. In this regard, the material 16 and/or the heating element 14 is not required to be attached to the cover 12. However, the material 16 and/or the heating element 14 can be integral to and/or attached to the cover 12.

FIGS. 3 and 4 show a sectional view of a heating apparatus 20 as applied to a body part 22 and wrapped around the body part 22, respectively, according to an embodiment of the present invention. In FIG. 3, a heating apparatus 20 is applied to at least a portion of a knee region 30 of an individual. In FIG. 4, the heating apparatus 20 is applied to at least a portion of a wrist region that extends into at least a portion of the forearm and/or arm region 32 of an individual 34. Preferably, the heating apparatus 20 is flexible and is able to conform to the contours of the body part 22. However, it should be appreciated that the heating apparatus 20 may be flat and essentially inflexible, or may be constructed in any suitable fashion. Coupled to the heating apparatus 20 is an external power source 24. It should be appreciated that the power source 24 may be internal and/or portable, and the energy may be supplied in any suitable manner. As shown in FIGS. 3 and 4, the heating apparatus 20 includes a cover 36 that incorporates a powered heating element 38 and a material 40 capable of emitting both for infrared radiation and negative ions. The heating apparatus 20 provides an effective amount of FIR/negative ion emission 26 into a body part 22. The amount of FIR/negative ion emission 26 should be sufficient to penetrate an effective depth into the body part 22 as shown in FIG. 3, the effective depth ranging anywhere from depths of conventional heating methods (approx. 2 mm to 3 mm) to depths approaching 40 mm or more achieved with FIR/negative ion technology. In a preferred embodiment, the depth of penetration is about 25 mm to 35 mm.

As previously discussed, the heating device or apparatus can be configured in any suitable manner to provide an effective amount of FIR and negative ions during heating. For example, FIG. 5 shows a cross-sectional view of a heating device 42 where the negative ion emission 44 and the FIR radiation 46 originate from the same material 40 according to an embodiment of the present invention. The heating device 42 is applied to a body part 22 wherein the heating device includes the cover 36 that surrounds the heating element 38 that is powered by an external power source 24 and the FIR and negative ion emitting material 40.

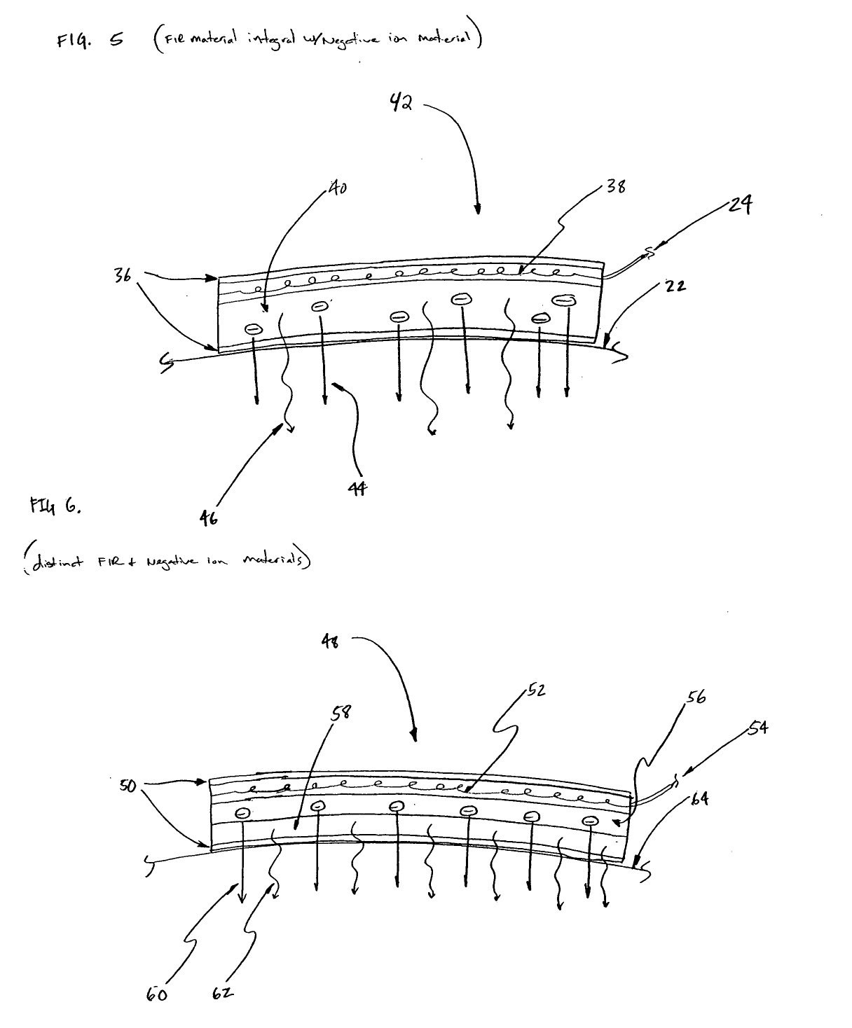

FIG. 6 shows a cross-sectional view of a heating apparatus 48 according to another embodiment of the present invention. In this embodiment of the present invention, a cover 50 is provided. The cover 50 can be made of any suitable material, preferably a substantially flexible material, such as a material made from a textile material suitable for applying heat, for example, to an individual during heat therapy. Coupled to one side of the cover 50 is a powered heating element 52. Coupled to the heating element 52 is an external power source 54. It should be appreciated that the power source 54 may be an internal and/or portable source, and the energy may be supplied in any suitable manner. In this embodiment, there is both a negative ion emitting material 56 and an FIR emitting material 58, both materials contained within the cover 50. The negative ion emitting material 56 and the FIR emitting material 58 provide an effective amount of negative ion emission 60 and FIR radiation 62, respectively, into a body part 64. The amount of negative ion emission 60 and FIR radiation 62 should be sufficient to penetrate an effective depth into the body part 64 for therapeutic application.

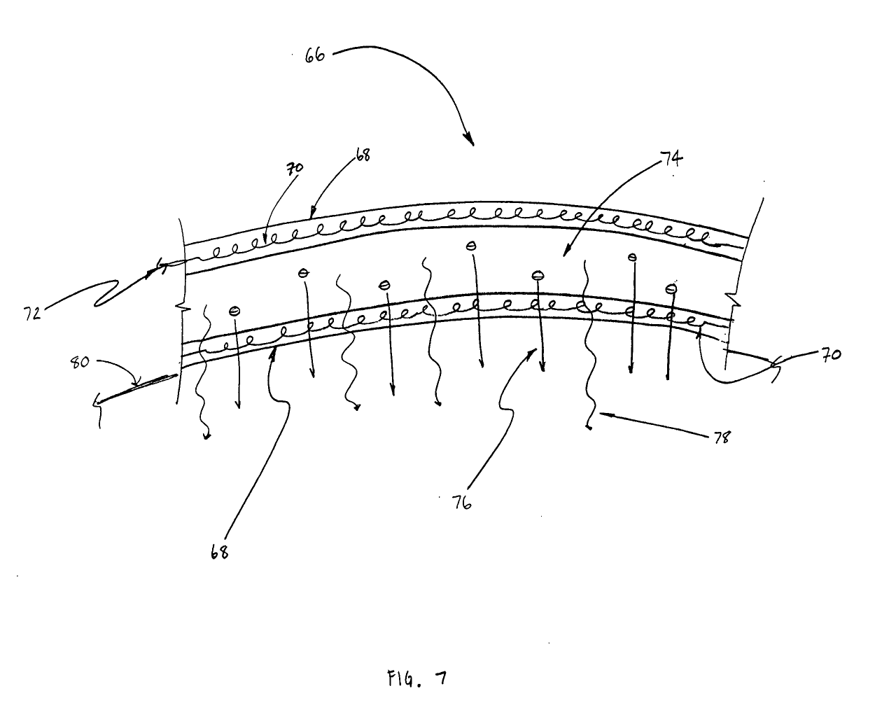

FIG. 7 shows a cross-sectional view of a heating apparatus 66 according to another embodiment of the present invention. In this embodiment of the present invention, a cover 68 is provided. The cover 68 can be made of any suitable material, preferably a substantially flexible material, such as a material made from a textile material suitable for applying heat. In this embodiment, the heating apparatus 66 includes a powered heating element 70 that is integral with the cover 68. Coupled to the heating element 70 is an external power source 72, as described in FIG. 6. In this embodiment, an FIR/negative ion emitting material 74 is adaptedly coupled to the cover 68 wherein the negative ion emission 76 and the FIR radiation 78 originate from the same material 79. The amount of negative ion emission 76 and FIR radiation 78 should be sufficient to penetrate an effective depth into the body part 80 for therapeutic application.

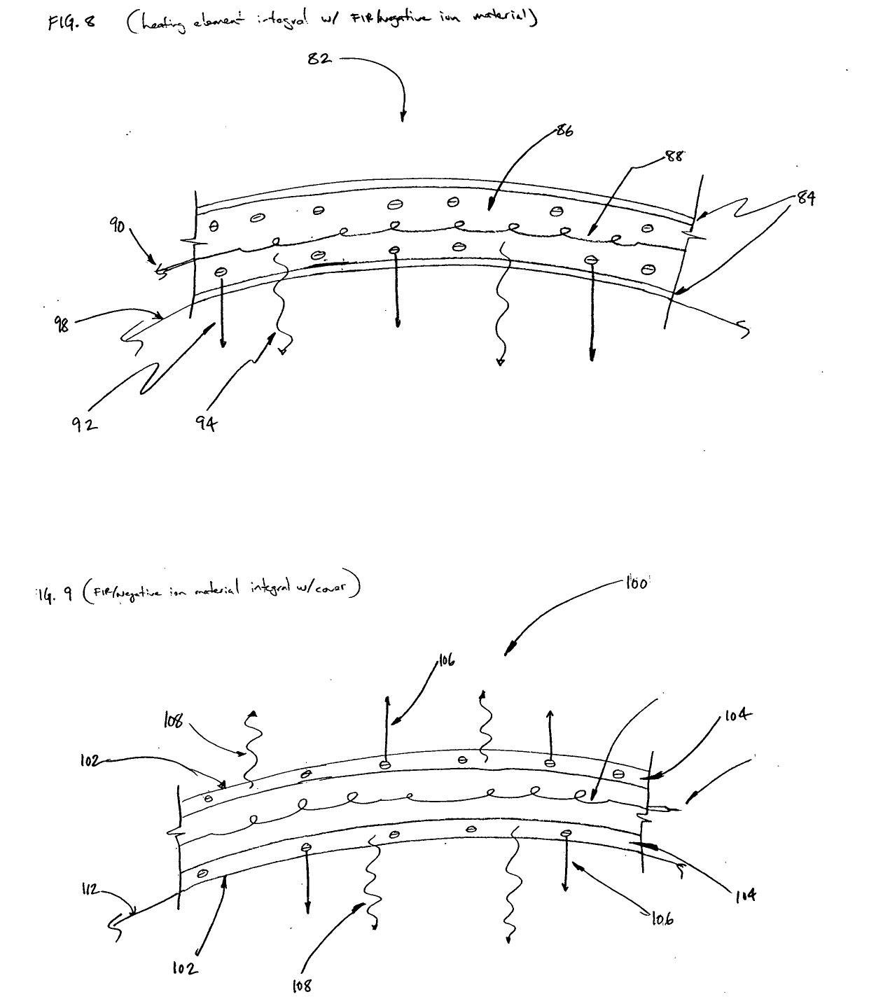

FIG. 8 shows a cross-sectional view of a heating apparatus 82 according to another embodiment of the present invention. In this embodiment of the present invention, a cover 84 is provided. An FIR/negative ion emitting material 86 is adaptedly coupled to the cover 84. In this embodiment, a powered heating element 88 is integral with the FIR/negative ion emitting material 86. Coupled to the heating element 88 is a power source 90, as described in FIG. 6. The negative ion emission 92 and the FIR radiation 94 originate from the same material. The amount of FIR/negative ion emission 96 should be sufficient to penetrate an effective depth into a body part 98 in a therapeutic manner.

FIG. 9 shows a cross-sectional view of a heating apparatus 100 according to another embodiment of the present invention. In this embodiment of the present invention, a cover 102 is provided. As shown, an FIR/negative ion emitting material 104 is integral with the cover 102. In this embodiment, the negative ion emission 106 and the FIR radiation 108 originate from the same material 104 and should be sufficient to penetrate an effective depth into a body part 112 during therapy or treatment of the body part 112. In this embodiment, a powered heating element 114 is adaptedly coupled to the cover 102. Coupled to the heating element 114 is a power source 116, as illustrated in FIG. 6.

It should be appreciated that the present invention can be configured in any suitable manner and with any suitable materials in addition to the embodiments described above. For example, the present invention can be configured as a heating pad, a heating wrap, a heating collar, a heating cuff, a heating patch, a heating blanket, other suitable configurations and combinations (not shown) thereof depending on the application, such as applying heat therapy to a specific body part or region of an individual in need thereof.

As applied, the present invention can be used in a variety of different and suitable applications, preferably during heat therapy to a person in need of same. In this regard, the present invention can be configured and constructed so as to be effectively applied to a variety of different body parts including, for example, an arm region, an elbow region, a knee region, a back region, a head region, a neck region, a foot region, a hand region, a forearm region, a shoulder region, a wrist region, an ankle region, the like and combinations thereof.

The heating devices and apparatuses of the present invention can be operated under any suitable condition so as to effectively apply heat therapy that includes emitting both far infrared radiation and negative ions. For example, the heating element can be operated at any suitable temperature or range thereof. In a preferred embodiment, the heating element is operated at a temperature ranging from about 55 degrees Celsius to about 65 degrees Celsius so as to cause the material to emit both far infrared radiation and negative ions in an effective and therapeutic manner.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A device capable of emitting both far infrared radiation and negative ions, the device comprising a powered heating element and a material adaptedly coupled to the powered heating element allowing the material to be effectively heated so as to emit both far infrared radiation and negative ions in a therapeutic manner.

2. The device of claim 1, wherein the powered heating element is integral with a cover that incorporates the powered heating element and the material.

3. The device of claim 1, wherein the powered heating element is integral with the material capable of emitting both far infrared radiation and negative ions.

4. The device of claim 1, wherein the material capable of emitting both far infrared radiation and negative ions is integral with a cover that surrounds the powered heating element.

5. The device of claim 1, wherein the material is selected from the group consisting of a single material having one or more constituents and combination of two or more materials.

6. The device of claim 1, wherein the material includes a first part including a bio-ceramic in an amount of about 95% by weight or less and a second part including a ceramic oxide in an amount of about 5% by weight or more.

7. The device of claim 6, wherein the first part comprises at least one compound selected from the group consisting of silicon oxide, aluminum oxide, iron oxide, magnesium oxide, derivatives thereof and combinations thereof.

8. The device of claim 6, wherein the ceramic oxide is selected from the group consisting of iron oxide, silicon oxide, titanium oxide, aluminum oxide, magnesium oxide, derivatives thereof and combinations thereof.

9. An apparatus capable of emitting both far infrared radiation and negative ions, the apparatus comprising:
a cover;
a powered heating element wherein the heating element is adaptedly coupled to the cover; and
a flexible material capable of emitting both far infrared radiation and negative ions wherein the flexible material is adaptedly coupled to the powered heating element and the cover allowing the material to be heated, and wherein the flexible material includes a first part including a bio-ceramic in an amount of about 95% by weight or less and a second part including a ceramic oxide in an amount of about 5% by weight or more.

10. The apparatus of claim 9, wherein the powered heating element is integral with the cover.

11. The apparatus of claim 9, wherein the powered heating element is integral with the flexible material.

12. The apparatus of claim 9, wherein the flexible material is integral with the cover.

13. The apparatus of claim 9, wherein the first part comprises at least one compound selected from the group consisting of silicon oxide, aluminum oxide, iron oxide, magnesium oxide, derivatives thereof and combinations thereof.

14. The apparatus of claim 9, wherein the ceramic oxide is selected from the group consisting of iron oxide, silicon oxide, titanium oxide, aluminum oxide, magnesium oxide, derivatives thereof and combinations thereof.

15. The apparatus of claim 9, wherein the apparatus is configured as a heating apparatus for therapeutic applications selected from the group consisting of a heating pad, a heating wrap, a heating collar, a heating cuff, a heating patch, a heating blanket, and combinations thereof.

16. A method of providing heat therapy to an individual in need of same, the method comprising the steps of:
providing a heating apparatus, the heating apparatus including a cover, a heating element, and a material capable of emitting both far infrared radiation and negative ions wherein the cover, heating element and the material are so constructed and arranged to form the heating apparatus;
applying the heating apparatus to at least a portion of a body part of the individual;
supplying power to the heating element; and
heating the material to release an effective amount of far infrared radiation and negative ions to provide heat therapy to the individual.

17. The method of claim 16, wherein the heating element is selected from the group consisting of a wire, a coil, a pad, a resistor, an exothermic chemical composition, and combinations thereof.

18. The method of claim 16, wherein the step of supplying power to the heating element includes at least one of an external power source and an internal power source.

19. The method of claim 16, wherein the body part is selected from the group consisting of an arm region, an elbow region, a knee region, a back region, a head region, a neck region, a foot region, a hand region, a forearm region, a shoulder region, a wrist region, an ankle region and combinations thereof.

20. The method of claim 16, wherein the step of releasing far infrared radiation and negative ions includes releasing an amount of far infrared radiation and negative ions sufficient to penetrate the body part to a depth ranging from about 2 cm to about 5 cm.

21. A method of manufacturing a heating apparatus, the method comprising the steps of:
providing a powered heating element and a material capable of emitting both far infrared radiation and negative ions; and
adaptedly coupling the powered heating element and the material to form the heating apparatus.

22. The method of claim 21, wherein the powered heating element is integrally formed with the material capable of emitting both far infrared radiation and negative ions.

23. The method of claim 21, wherein the powered heating element is integrally formed with a cover that incorporates the material.

24. The method of claim 21, wherein the material capable of emitting both far infrared radiation and negative ions is integrally formed with a cover that incorporates the powered heating element.

25. The method of claim 21, wherein the material is selected from the group consisting of a single material having one or more constituents and combination of two or more materials.

26. The method of claim 21, wherein the material includes a first part including a bio-ceramic in an amount of about 95% by weight or less and a second part including a ceramic oxide in an amount of about 5% by weight or more.

27. The method of claim 26, wherein the first part comprises at least one compound selected from the group consisting of silicon oxide, aluminum oxide, iron oxide, magnesium oxide, derivatives thereof and combinations thereof.

28. The method of claim 26, wherein the ceramic oxide is selected from the group consisting of iron oxide, silicon oxide, titanium oxide, aluminum oxide, magnesium oxide, derivative thereof and combinations thereof.
